(19) United States
(12) Patent Application Publication
(10) Pub. No.: US 2016/0106654 A1
(43) Pub. Date: Apr. 21, 2016

(54) ADDITIVE OR SYNERGISTIC SUNSCREEN, ANTIOXIDANT, AND DNA REPAIR ENZYME COMPOSITION

(71) Applicant: US CosmeceuTechs, LLC, Richmond, VA (US)

(72) Inventors: Joseph Abernathy Lewis, II, Chesterfield, VA (US); Arthur J. Pellegrino, Newtown, PA (US); Lavinia C. Popescu, Jackson Heights, NY (US); Jose Ricardo Fernandez, Franklin Park, NJ (US); Eduardo Perez, Somerset, NJ (US); Paul A. Sowyrda, MEDFIELD, MA (US)

(21) Appl. No.: 14/886,111

(22) Filed: Oct. 19, 2015

Related U.S. Application Data
(60) Provisional application No. 62/066,425, filed on Oct. 21, 2014.

(51) Int. Cl. A61K 8/66; A61K 8/49; A61K 8/46; A61Q 17/04

(52) U.S. Cl. CPC A61K 8/66; A61Q 17/04; A61K 8/4946; A61K 8/46

(57) ABSTRACT

A composition having an sunscreen agent, an antioxidant, and a DNA repair enzyme provides a beneficial and/or synergistically improved effect in reducing mutagenic formations, particularly in topically applied formulations.

### CROSS-REFERENCE TO RELATED APPLICATIONS

The present application claims priority to U.S. Appl. Ser. No. 62/066,425, filed Oct. 21, 2014, which is incorporated in its entirety herein.

### STATEMENT REGARDING PRIOR DISCLOSURES BY THE INVENTOR

Applicants arranged for and authorized a scientific study of the effects of antioxidant compositions commercially available and allowed this information to be published as Emanuele, E.; Spencer, J. M.; Braun, M. “An Experimental Double-Blind Irradiation Study of a Novel Topical Product (TPF 50) Compared to Other Topical Products with DNA Repair Enzymes, Antioxidants, and Growth Factors With Sunscreens: Implications for Preventing Skin Aging and Cancer” J. Drugs Dermatol. 2014, 13(3), 611-617, in early March of 2014. This reference is incorporated herein in its entirety.

Applicants furthermore allowed for several press-releases of the break-through results of their study, all having content within the scope of a release entitled “Elizabeth Arden Unveils a New Standard in Topical Skincare Protection Triple Protection Factor Broad Spectrum Sunscreen SPF 50+ as Part of New Professional Skincare Line, Elizabeth Arden Rx,” on the newswire on and after March 4, 2014.

### FIELD

The invention pertains to antioxidant and DNA repair enzyme compositions, particularly to compositions having at least one sunscreen component, typically measured by the sun protection factor (SPF) scale.

### BACKGROUND

Exposure of mammalian skin to sunlight, particularly the ultraviolet (UV) wavelengths, can cause sunburn (erythema) and blistering (edema). Exposure to UV light can also cause the skin to feel dry and taut in moderate doses, and to peel if exposed to higher doses. However, less readily discernible acute effects, such as photo-immunosuppression, cross-linking of deoxyribonucleic acid (DNA), formation of sunburn cells, and loss of Langerhans cells, and more serious long term effects, such as skin cancer and premature aging of the skin, also result from exposure to sunlight. Amongst these, particular concerns are the formation of DNA and protein photolesions that can give rise to photoaging, melanoma, and non-melanoma skin cancers (NMSC).

Traditional sunscreen products aim to protect the skin from some of the harmful effects of UV light exposure. These products contain molecules that absorb, block, scatter, and/or reflect harmful wavelengths of UV light before they can reach the skin. The absorbed UV light is converted to heat energy and rapidly dissipated to the skin and environment, which allows these molecules to revert to a lower energy state, and subsequently absorb another photon of light. In this manner, sunscreen agents can absorb numerous photons of ultraviolet light in a relatively short period of time. By absorbing the harmful wavelengths of light, sunscreen products prevent many of the acute and chronic effects caused by ultraviolet light.

The effectiveness of sunscreen products is expressed as a Sun Protection Factor (SPF) value. SPF is a measure of a topical protective capacity of a substance such as a sunscreen against type B ultraviolet radiation (UV-B). The higher the SPF, the more protection a sunscreen offers against UV-B radiation, which is the radiation that causes sunburn. SPF labeling of sunscreen products is regulated by the U.S. Food and Drug Administration (FDA) and other regulatory agencies on an international basis. SPF is an example of a standardized sun protection factor for a substance. Determination of SPF values of products is described in Title 21 of the Code of Federal Regulations, specifically at 21 C.F.R. §352.73, which is incorporated by reference herein.

Mammal, particularly human, skin is known to be damaged and aged by exposure to type A ultraviolet (UV-A) radiation, which does not cause sunburn. For example, UV-A radiation may cause DNA damage to cells which may increase the risk of malignant melanomas. Examples of substances that provide protection against UV-A radiation include zinc oxide, oxybenzone, avobenzone, and meroxyl.

Common commercially-available suncare products are emulsions or suspensions with SPF values ranging from 2 to 50+ (up until 2012 in the US, SPF values of greater than SPF 50 were allowed), often including ethylhexyl methoxycinnamate, octyl salate, oxybenzone, zinc oxide, or titanium dioxide as the sunscreen agent(s).

Examples of standards that have been developed for measuring the topical protective capacity of a substance against UV-A radiation include Critical Wavelength (USA/FDA Monograph for “Broad Spectrum” claim requirement) Persistent Pigment Darkening (PPD), Immediate Pigment Darkening (IPD), Boots Star System, and the Japanese PA system. These measurements are further examples of a sun protection value of a substance.

Another SPF rating designation, sometimes referred to as UPF (ultra-violet protection factor), measures the topical protective capacity of a substance against both UV-A and UV-B radiation. Many suncare products claim to help prevent skin cancer and premature aging of the skin, including providing “broad spectrum” protection against both UVA and UVB radiation. The UVB wavelengths of the solar ultraviolet spectrum (280 to 315 nm) are more efficient at producing a sunburn but do not penetrate the skin very deeply. The UVA wavelengths (315 to 400 nm) of the solar ultraviolet spectrum are at least 10 times less effective at producing a sunburn, but readily penetrate the entire depth of the skin where a different kind of damage can occur, including structural damage to the dermal fibroblasts and dermal proteins such as collagen and elastin. Higher SPF products usually incorporate both UVB and UVA sunscreen agents into the formulation, in which case the formulation is designated as a broad spectrum sunscreen; sunscreens in the US are now required to provide broad spectrum UV protection.

In addition, longer radiation wavelengths, including Infrared (IR) have been shown penetrate the skin even deeper also resulting in production of toxic free radicals that damage the skin. IR radiation represents over 50% of the radiation arriving at the earth's surface; currently US & INTL sun protection standards do not address the need for protection against IR radiation. In addition there are known facts about the deficiencies of SPF skin protection. The benefits of sunscreen have been studied for decades. Studies show that in the recommended quantity of cream: SPF 15 blocks 94% of UVB radiation; SPF 30 blocks 97% of UVB radiation; and SPF 45 blocks 98% of UVB radiation.

However, it is known that sunscreen only stops between 45% and 55% of free radicals induced by UVA radiation. Haywood, R. et al. “Sunscreens Inadequately Protect Against Ultraviolet-A-Induced Free Radicals in Skin: Implications for Skin Aging and Melanoma?” J. Invest. Dermatol. 2003, 121(4), 862-868. Research has revealed that most people apply only 25-50% of the recommended amount of sunscreen. Neale, R. et al. “Application Patterns Among Participants Randomized to Daily Sunscreen Use in a Skin Cancer Prevention Trial” Arch. Dermatol. 2002, 138(10), 1319-1325. Moreover, slightly more than half of the energy from the sun arrives on earth in the form of infrared radiation, and infrared A penetrates the skin deeper than UVA/UVB causing free radical formation and accelerated skin aging. Schroeder, P. et al. “Infrared A Radiation Effects on the Skin” Piel 2011, 26(6), 259-262. Sunscreens are not antioxidants, and therefore cannot scavenge toxic free radicals once they are formed and provide no protection against air pollution, particulate pollution, cigarette smoke, ozone, or other sources of exogenous free radicals.

Damage to human skin can also be caused by oxidative stress from external environmental stressors, including sunlight radiation, but also from other non-radiation sources such as exposure to air pollution, cigarette smoke, chemicals, cosmetics, drugs, ozone, and even oxygen itself. In addition to externally generated oxidative stress, internally generated oxidative stress may occur as a natural by-product of cellular energy production. Both internal and external oxidative stress leads to inflammatory pathways mediated by the formation of free radicals (molecules with unpaired electrons that are highly reactive) that, left unchecked, can cause severe cellular damage to cell membranes, lipids, proteins and DNA.

It is known that ultraviolet light has the capacity to create singlet oxygen from normal atmospheric oxygen (also known as triplet oxygen). Since molecular oxygen contains two unpaired electrons in its normal state, the effect of ultraviolet light upon triplet oxygen results in the creation of a more reactive oxygen species (i.e., singlet oxygen). In the singlet state, the oxygen molecule is capable of reacting with a variety of molecules that it would not react with in its normal triplet state.

Singlet oxygen can abstract a hydrogen atom from many different molecules creating other free radicals in addition to the superoxide radical from the oxygen molecule. Both of these radicals are extremely reactive with a variety of molecules naturally present in and on the skin, and can decompose to form hydroxy radicals, which are also extremely reactive in the presence of molecules in and on the skin. The superoxide radical, also a natural byproduct of metabolic energy production, causes serious deleterious effects to living cells if not quenched, neutralized, or reduced almost immediately after production. It is known that lipid peroxidation is a major problem in biological systems. Protecting against cell membrane oxidation is of paramount importance in living biological systems since the cell membrane is the cell's first line of defense against oxidation.

The lifetime summation of damage caused by run-away free radicals is one of the main theories of aging, sometimes referred to as “the damage accumulation theory of aging.” There is a strong interest in modern medicine regarding the use of antioxidants, substances that scavenge and eliminate free radicals, to counter the deleterious effects (i.e., aging and non) of the free radical mediated inflammatory pathways caused by oxidative stress. DNA damage has become a research focus of UV-associated skin aging and carcinogenesis, since DNA mutation plays an important in skin aging and cancer.

Research has indicated that the formation of helix-distorting photoproducts such as cyclobutane pyrimidine dimers (CPD), as well as oxidative damage to DNA bases, including the formation of 8-oxo-7,8-dihydro-2′-deoxyguanosine (8OHdG) are among the key DNA lesions associated with photoaging and tumorigenesis. Besides DNA lesions, UV radiation-induced formation of free radicals can result in protein carbonylation (PC), a major form of irreversible damage that can biologically inactivate proteins. In addition to protein carbonylation, protein glycation, or non-enzymatic glycosylation, is a manner in which proteins can become damaged or inactivated.

To deal with the problems arising from photo-induced DNA damage, the topical application of DNA repair enzymes has been proposed, thereby enhancing DNA repair synthesis. See Kibitel, J. T., et al. Photochem. Photobiol. 1991, 54(5), 743-760; and Yarosh, D. B., et al. J. Invest. Dermat. 1991, 97(1), 147-150. It has also been demonstrated that different xenogenic DNA repair enzymes (photolyase, endonuclease, and 8-oxoguanine glycosylase [OGG1]) are useful for reversing UV-induced DNA damage in human skin when applied topically in liposomal form. Emanuele, E., et al. J. Drugs. Dermatol. 2013, 12, 1017-1021; Spencer, J. M., et al. J. Drugs Dermatol. 2013, 12, 336-40; Stege, H. et al. J. Photochem. Photobiol. B. 2001, 65, 105-108; Stege, H., et al. Proc. Natl Acad. Sci. 2000, 97, 1790-1795. Photolyase plus sunscreens has been shown to be superior to sunscreens alone in reducing the formation of cyclobutane pyrimidine dimers (CPD) in irradiated human skin. Berardesca, E., et al. Mol. Med. Rep. 2012, 5(2), 570-574.

Furthermore, U.S. Pat. No. 6,015,548 (Siddiqui) has proposed a formulation in which an antioxidant is combined with a sunscreen agent, reporting this combination to give unexpectedly superior protection of the skin against the detrimental effects caused by exposure to ultraviolet radiation. Siddiqui describes that antioxidants substances, including free radical absorbers or scavengers, prevent oxidation processes, including oxidation of a molecule such as a lipid, lipoprotein, protein, or DNA, or autooxidation of fats containing unsaturated compounds. Siddiqui describes antioxidants used in the field of cosmetics and pharmacy to include, for example, Vitamin C and Vitamin E (and their esters), magnesium ascorbyl phosphate, panthenol, beta glucan, grape seed extract, superoxide dismutase, kinetin, ubiquinone, ascorbic acid, lipoic acid sesamol, colic acid derivatives, butylhydroxy anisole, and butylhydroxy toluene. Siddiqui describes that antioxidants can help protect human cells, such as skin cells, from both externally and internally generated oxidative stress. However, Siddiqui is silent on a DNA (or other biomolecule) repair enzyme in its composition.

There remains a need in the market for an improved means of reducing and/or preventing cellular injuries and diseases related to UV-exposure and other forms of oxidative stress.

### SUMMARY

No references known to us indicate that combining the preventative and/or remedial approaches from the known, separate formulations would even function in an additively positive manner. Likewise, there is no suggestion in the art that such a combination would produce a synergistic effect in the protection of the skin against non-melanoma skin cancer (NMSC), stress damage, and/or aging, and as measured by critical biomarkers associated with these skin conditions. Surprisingly, therefore, it has now been found that addition of both a unique blend of one or more DNA and/or protein protection antioxidants, e.g., a Protein Protection Antioxidant Complex (PPAC) and a blend of three or more DNA repair enzymes (this term being meant to include DNA, protein, RNA, etc., repair enzymes—DNA repair enzymes complex) to traditional sunscreens can add positive additive, synergistic, and/or multiplicative value against stress damage markers when applied topically to human skin in vivo.

An aspect of the invention provides a composition, comprising: a sunscreen agent; a plurality of DNA repair enzymes; and a plurality of DNA and/or protein protective antioxidants.

### BRIEF DESCRIPTION OF THE DRAWINGS

Aspects of certain embodiments of the invention, to which the claims are not limited, are illustrated in the attached figures, which include:

FIG. 1 shows the results of a comparison of components of the inventive composition against the total composition in preventing the formation of cyclobutane pyrimidine dimers (CPD); and

FIG. 2 shows the results of a comparison of components of the inventive composition against the total composition in preventing the formation of 8-oxo-7,8-dihydro-2′-deoxyguanosine (8OHdG) DNA lesions; and

FIG. 3 shows the results of a comparison of components of the inventive composition against the total composition in preventing protein carbonylation (PC) by UV radiation-induced free radicals;

FIG. 4 shows the results of a comparison of components of the inventive composition against the total composition in preventing CPD, 8OHdG, and PC; and

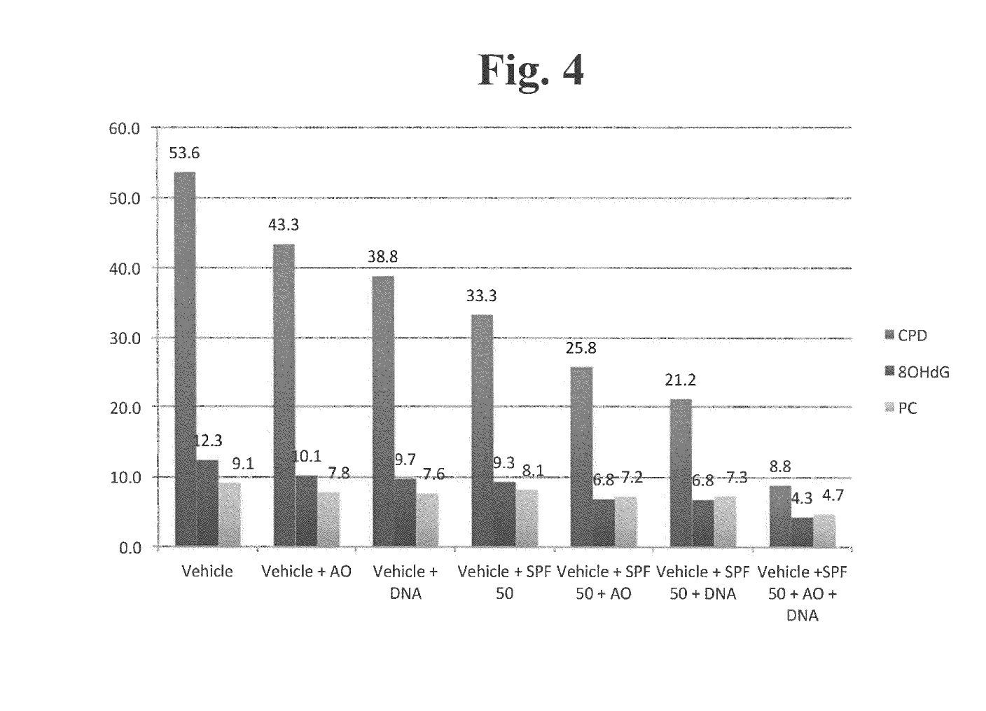

FIG. 5 shows an artistic rendition of the effect of various components regarding three levels of protection, in a manner indicated, of the inventive composition.

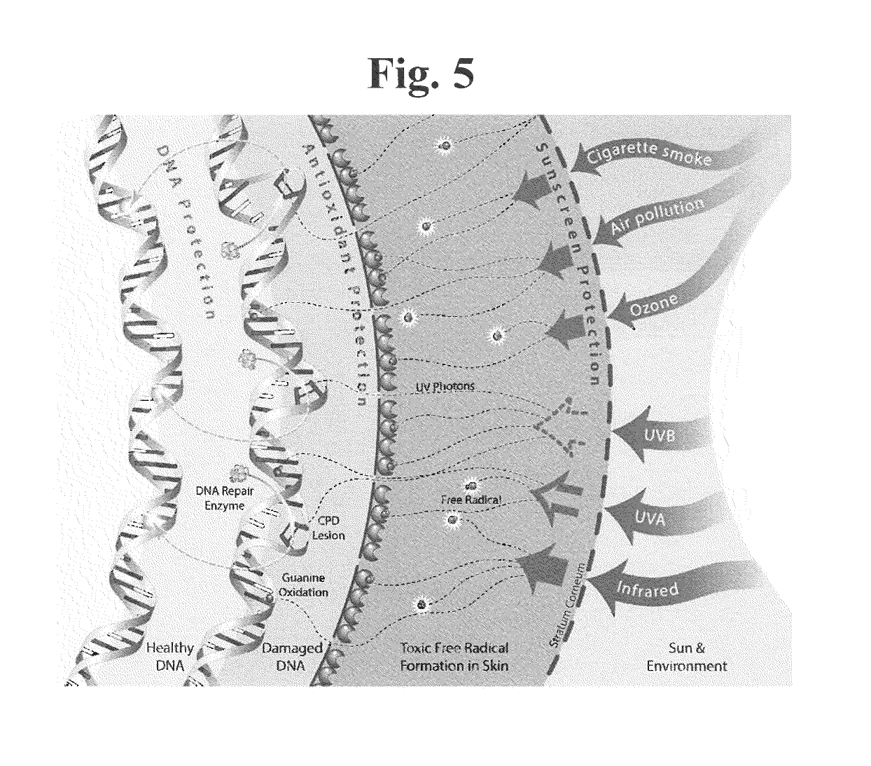

### DETAILED DESCRIPTION

An aspect of the invention provides a composition, comprising: a sunscreen agent; a DNA repair enzyme (this term being meant to include one or more DNA, protein, RNA, etc., repair enzymes), preferably a DNA repair enzyme complex comprising a plurality of DNA repair enzymes; and an antioxidant, preferably an antioxidant complex comprising a plurality of antioxidants, more specifically those antioxidants with an affinity for protecting DNA and/or proteins.

An aspect of the invention provides a composition with one or more sunscreen agents, one or more DNA repair enzymes, and one or more antioxidants, which are preferably DNA and/or protein protection antioxidants. The composition may contain one, two, or more sunscreen agents, two or more DNA repair enzymes, and two or more DNA and/or protein protection antioxidants; or one, two, or more sunscreen agents, three or more DNA repair enzymes, and three or more DNA and/or protein protection antioxidants. Such compositions can provide an additive benefit for reducing molecular biomarkers for pre-mature skin aging and non-melanoma skin cancers (NMSC). Such a composition can also have a multiplicative benefit for reducing molecular biomarkers for pre-mature skin aging and non-melanoma skin cancers (NMSC). An aspect of the invention provides a synergistic improvement in protection when topically applied to human skin.

The sunscreen agent according to the invention is preferably one or more of a para-aminobenzoic acid, a salt of para-aminobenzoic acid, a derivative of para-aminobenzoic acid (e.g., octyl para-N,N-dimethylaminobenzoate, “padimate O”), ethylhexyl methoxycinnamate, DEA methoxycinnamate, ethylhexyl salicylate, 3,3,5-trimethylcyclohexyl 2-hydroxybenzoate (“homosalate”), tris(2-hydroxyethyl)ammonium 2-hydroxybenzoate (“trolamine salicylate”), TEA salicylate, 2-hydroxy-4-methoxyphenyl)-phenylmethanone (“oxybenzone”), (2-hydroxy-4-methoxyphenyl)-(2-hydroxyphenyl)methanone (“dioxybenzone”), 4-hydroxy-2-methoxy-5-(oxo-phenylmethyl)-benzenesulfonic acid (“sulisobenzone”), 1-(4-methoxyphenyl)-3-(4-tert-butylphenyl)propane-1,3-dione (“avobenzone”), 2-ethylhexyl 2-cyano-3,3-diphenyl-2-propenoate (“octocrylene”), (1R,2S,5R)-2-isopropyl-5-methylcyclohexyl 2-aminobenzoate (“menthyl anthranilate”), propyl esters of gallic acid, octyl esters of gallic acid, dodecyl esters of gallic acid, butylated hydroxyanisole (isomerically pure or as a mixture of ortho and meta isomers), butylated hydroxytoluene, nordihydroguaiaretic acid, talc, kaolin, chalk, precipitated silica, and/or one or more inorganic pigments (oxides of titanium, zinc, zirconium, silicon, iron, manganese, and/or cerium). Mixtures of these components are specifically contemplated, as are mixtures of any members of subgenuses listed. Further general classes of suitable sunscreen agents according to the invention are those set forth in U.S. Appl. Ser. No. 62/066,425, in part overlapping with the above.

The sunscreen agent according to the invention may be present in the composition in an amount of anywhere from 0.1 to 40 wt. %, 1 to 35 wt. %, 2 to 20 wt. %, 4 to 15 wt. %, 5 to 10 wt. %, or 5.5 to 8 wt. %, relative to a total mass of the composition. A weight of any sunscreen agent may be any whole, half, quarter, tenth, twentieth unit (or any combination of these partial units) of any of 1, 2, 3, 4, 5, 6, 7, 8, 9, or 10, i.e., 0.05, 0.1, 0.15, 0.2, 0.25, 0.3, 0.35, 0.4, 0.45, 0.5, . . . , 39.9, 39.95, 40 wt. %. The total weight of all sunscreen agents together in the formulation may be within any of the above mentioned ranges, i.e., the total of all sunscreen agents in the formulation may be from 0.1 to 40 wt. %, etc. The total of all sunscreen agents together is preferably greater than 1 wt. %, preferably at least 2.5 wt. %, preferably at least 5 wt. %, preferably greater than 7.5 wt. %, most preferably greater than 10 wt. %, relative to the total weight of the formulation.

The sunscreen agent according to the invention may be in such a form so as to impart a sun protection factor (SPF) of anywhere from 1 to 50 or above, e.g., 75 or 100. Typically, the SPF of the formulation will be 2, 5, 10, 15, 20, 25, 30, 35, 40, 45, 50, or greater than 50. In some embodiments, the SPF of the formulation may be 30, 35, 40, 45, 50, or greater than 50. In certain embodiments, however, lower SPF formulations may be desired. The SPF of the formulation may be in a range having any of these values as upper or lower endpoints, e.g., 1-100, 25-75, 2-50, 15-50, 5-10, 2-35, 30-50, 20-45, 35-75, 40-50, 25-50, etc.

The DNA repair enzyme according to the invention is preferably one or more of photolyase, endonuclease, 8-oxoguanine glycosylase, alkylase (e.g., alkylguanine-DNA alkyl transferase), or a mixture thereof. Mixtures of these components are specifically contemplated and preferred in certain embodiments. The enzymes may be endogenic or exogenic, and may be plant, animal, fungal, protozoan/protist, yeast, archaeal, and/or bacterial in derivation (e.g., human, yeast, algae, plankton, etc.), and may be a synthetic variant thereof or a combination of natural and synthetic species. The DNA repair enzyme may include any enzyme or combination of enzymes of the DNA glycosylase family, including E. coli AlkA, S. cerivisiae Mag1, and human MPG enzymes; E. coli UDG, S. cerivisiae Ung1, and human UNG enzymes; E. coli Fpg, S. cerivisiae Ogg1, and human hOGG1 enzymes; E. coli Nth, S. cerivisiae Ntg1 and/or Ntg2, and human hNTH1 enzymes; E. coli Nei and human hNEIL1, hNEIL2, and/or hNEIL3 enzymes; E. coli MutY and human hMYH enzymes; and human hSMUG1, TDG, and MBD4 enzymes. Separately, or in addition to any of the preceding family, the DNA repair enzyme may include one or more apurinic/apyrimidinic (AP) endonucleases, of any of classes I, II, II, and/or IV, e.g., human AP endonuclease APE1, APEX1, APEX2, EndoIV, and ExoIII enzymes. Besides or further to these, human (or corresponding plant, bacterial, fungal, protozoan, etc.) DNA repair enzymes set forth in U.S. Appl. Ser. No. 62/066,425, may be used in the inventive composition.

The DNA repair enzyme raw material composition may contain various naturally derived DNA repair enzymes, such as photolyase (e.g., from plankton extract), 8-oxogaunine glycosylase (e.g., from Arabidopsis thaliana extract and/or mustard seed extract), and/or endonuclease (e.g., from one or more micrococcus lysates). Some useful enzyme compositions are marketed under the trade names of the PHOTOSOMES™ composition (containing plankton extract, lecithin, water, and phenoxyethanol), which may be used, e.g., in 0.001 to 40 wt. %, 0.005 to 20 wt. %, 0.01 to 10 wt. %, 0.05 to 8 wt. %, or 0.1 to 5.0 wt. % of a composition; the ROXISOMES™ composition (containing Arabidopsis extract, lecithin, and phenoxyethanol), which may be used, e.g., in 0.001 to 40 wt. %, 0.005 to 20 wt. %, 0.01 to 10 wt. %, 0.05 to 8 wt. %, or 0.1 to 5.0 wt. % of a composition; and the ULTRASOMES™ composition (containing UV-endonuclease from Micrococcus luteus or tetragenus lysate, lecithin, and water), which may be used in 0.001 to 40 wt. %, 0.005 to 20 wt. %, 0.01 to 10 wt. %, 0.05 to 8 wt. %, or 0.1 to 5.0 wt. % of a composition. The DNA repair enzyme or combination may be present in a liposome encapsulated delivery system.

A preferred embodiment uses at least one of photolyase, endonuclease (e.g., T4 endonuclease), 8-oxoguanine glycosylase, alkylase (e.g., alkylguanine-DNA alkyl transferase). Combinations including enzyme extracts from two or more species of any one, two, or three of photolyase, endonuclease, and 8-oxoguanine glycosylase are preferred. A particularly preferred embodiment of the invention uses a DNA repair enzyme comprising each of photolyase, endonuclease, and 8-oxoguanine glycosylase.

The DNA repair enzyme raw material according to the invention may be present in the composition in an amount of anywhere from 0.1 to 5.0 wt. %, preferably 0.3 to 4.0 wt. %, more preferably 0.3 to 2.5 wt. %, 0.3 to 2.0 wt. %, 0.3 to 1.5 wt. %, even 0.3 to 1.0 wt%, relative to a total mass of the composition. A source of such raw material may be, for example, the PHOTOSOMES™ composition, the ROXISOMES™ composition, and/or the ULTRASOMES™ composition. A weight of any DNA repair enzyme raw material may be any whole, half, quarter, tenth, twentieth unit (or any combination of these partial units) of any of 1, 2, 3, 4, or 5, i.e., 0.05, 0.1, 0.15, 0.2, 0.25, 0.3, 0.35, 0.4, 0.45, 0.5, . . . , 4.9, 4.95, 5 wt. %. Each DNA repair enzyme, preferably naturally derived, may be present at an concentration of from greater than 0 wt. % to less than or equal to 1.0 wt. %, preferably from greater than 0 wt. % to less than or equal to 0.5 wt. %, from greater than 0 wt. % to less than or equal to 0.1 wt. %, relative to a total composition weight. For example, each DNA repair enzyme may be present in an amount of greater than or equal to 10⁻⁹, 10⁻⁸, 10⁻⁷, 10⁻⁶, 10⁻⁵, 10⁻⁴, or 10⁻³ wt. %, greater than or equal to 0.005 wt. %, greater than or equal to 0.01, greater than or equal to 0.015 wt. %, greater than or equal to 0.02 wt. %, greater than or equal to 0.025 wt. %, . . . greater than or equal to 0.095 wt. %, up to and including 0.1 wt. %. The DNA repair enzyme may further be present, but present only in an amount of less than 0.1 wt. %, less than or equal to 0.095 wt. %, less than or equal to 0.09 wt. %, less than or equal to 0.085 wt. %, . . . less than or equal to 0.025 wt. %, less than or equal to 0.02 wt. %, less than or equal to 0.015 wt. %, less than or equal to 0.01, less than or equal to 0.005 wt. %, less than or equal to 10⁻³, 10⁻⁴, 10⁻⁵, 10⁻⁶, 10⁻⁷, 10⁻⁸, or 10⁻⁹ wt. %, etc. The total weight of all DNA repair enzymes together in the formulation may be within any of the above mentioned ranges.

The DNA repair enzymes are preferably selected from those which counteract and/or repair cyclobutane pyrimidine dimer (CPD) formation, 8-oxo-7,8-dihydro-2′-deoxyguanosine (8OHdG) formation, and/or nucleotide alkylation. In addition, a system of evaluation of protection provided by one or more antioxidants may be that described in our own work, US 2006/0171886 A1, which is incorporated by reference herein, optionally modified to include a DNA damage analysis, based preferably on the specific markers cyclobutane pyrimidine dimer (CPD) and/or 8-oxo-7,8-dihydro-2′-deoxyguanosine (8OHdG) formation. Evaluation results of DNA repair protection for certain antioxidant substances are shown in Table 1.

TABLE 1
Normal human epidermal keratinocytes (NHEK), UVB, and DNA damage
analysis assay (5 μg/mL, n = 2)

| Compound | CPD inhibition (%) | 8-oxoG inhibition (%) |
|---|---|---|
| Chlorogenic Acid | 36.0 | 52.5 |
| Ferulic Acid | 15.7 | 41.1 |
| L-Ascorbic Acid | 33.1 | 44.1 |
| Epigallocatechin Gallate | 44.5 | 41.9 |
| Resveratrol | 51.9 | 40.6 |
| Quercetin | 45.7 | 42.4 |
| L-Carnosine | 51.6 | 45.0 |

A preferred embodiment of the invention uses a protein protection antioxidant comprising N-acetyl-S-farnesyl-L-cysteine (N-acetyl-S-(3,7,11-trimethyl-2E,6E,10-dodecatrienyl)-L-cysteine), L-carnosine, L-ergothioneine, or a mixture thereof. A particularly preferred embodiment of the invention uses a protein protection antioxidant comprising N-acetyl-S-farnesyl-L-cysteine, L-carnosine, and L-ergothioneine.

The antioxidant(s) are preferably selected from those which protect against and/or prevent protein carbonylation (PC), protein glycation, lipidation, lipid glycation, protein alkylation, and/or lipid alkylation. Our system of evaluation from US 2006/0171886 A1, which may be modified to include a protein carbonylation, protein glycation, and/or protein alkylation analysis, may be used to evaluate the effectiveness of substances. Test results for selected antioxidants in the protection of human skin against protein carbonylation are shown below in Table 2.

TABLE 2
Normal human epidermal keratinocytes (NHEK), UVB, and
protein carbonylation assay (5 μg/mL, n = 2)

| Compound | PC inhibition (%) |
|---|---|
| Chlorogenic Acid | 0 |
| Ferulic Acid | 0 |
| L-Ascorbic Acid | 87.2 |
| Epigallocatechin Gallate | 100 |
| Resveratrol | 0 |
| Quercetin | 0 |
| L-Carnosine | 100 |
| L-Ergothioneine | 100 |

A combined score of protection against protein carbonylation, repair of DNA damaged by cyclobutane pyrimidine dimer (CPD) formation, and repair of DNA damaged by 8-oxo-7,8-dihydro-2′-deoxyguanosine (8OHdG) formation is shown below in Table 3.

TABLE 3
Protection measured by repair of DNA damaged
by cyclobutane pyrimidine dimer (CPD) and
8-oxo-7,8-dihydro-2′-deoxyguanosine (8OHdG) formation,
and protein carbonylation (normalized for concentration)

| Compound | CPDs Score | 8oxo Score | PC Score | Total Score* |
|---|---|---|---|---|
| L-Carnosine | 18 | 17 | 17 | 52 |
| Resveratrol | 17 | 18 | 12 | 47 |
| L-Ergothioneine | 17 | 14 | 16 | 47 |
| Ferulic Acid | 17 | 19 | 10 | 46 |
| α-Tocopherol | 16 | 18 | 12 | 46 |
| Chlorogenic Acid | 18 | 20 | 7 | 45 |
| Ascorbic Acid | 16 | 20 | 0 | 36 |
| EGCG | 15 | 18 | 3 | 36 |

*a maximum total score is 60

An antioxidant according to the invention is preferably a DNA and/or protein protective antioxidant, i.e., an antioxidant shown to substantially protect proteins as measured by at least 15, 20, 25, 30, 35, 40, 45, 50%, or greater inhibition of carbonylation, glycation, and/or other forms of alkylation. The protein protection antioxidant preferably includes one or more of N-acetyl-S-farnesyl-L-cysteine (N-acetyl-S-(3,7,11-trimethyl-2E,6E,10-dodecatrienyl)-L-cysteine), L-ergothioneine, 2-allyl-pyrroloquinoline quinone, L-carnosine, quercetin, resveratrol, epigallocatechin gallate, L-ascorbic acid, ferulic acid, and chlorogenic acid. Preferably, the antioxidant includes one or more of L-ergothioneine, L-carnosine, quercetin, resveratrol, epigallocatechin gallate, ferulic acid, chlorogenic acid, and N-acetyl-S-farnesyl-L-cysteine. A particularly preferred embodiment of the invention uses a protein protection antioxidant comprising N-acetyl-S-farnesyl-L-cysteine, L-carnosine, and L-ergothionine.

The protein protection antioxidant may be present in the composition in an amount of anywhere from 0.0001 to 20.0 wt. %, 0.001 to 10.0 wt. %, 0.01 to 7.5 wt. %, 0.1 to 5.0 wt. %, preferably 0.25 to 3.5 wt. %, more preferably 0.5 to 3.0 wt. %, even 1.0 to 2.5 wt. %, relative to a total mass of the composition. A weight of any protein protection antioxidant may be any whole, half, quarter, tenth, twentieth unit (or any combination of these partial units) of any of 1, 2, 3, 4, or 5, i.e., 0.05, 0.1, 0.15, 0.2, 0.25, 0.3, 0.35, 0.4, 0.45, 0.5, . . . , 4.9, 4.95, 5 wt. %. The total weight of all protein protection antioxidants together in the formulation may be within any of the above mentioned ranges.

A preferred embodiment of the invention provides a composition comprising: a sunscreen agent comprising any common sunscreen agent; a protein protection antioxidant comprising quercetin, resveratrol, epigallocatechin gallate, L-ascorbic acid, ferulic acid, chlorogenic acid, N-acetyl-S-(3,7,11-trimethyl-2E,6E,10-dodecatrienyl)-L-cysteine, L-carnosine, and/or L-ergothionine; and a DNA enzyme repair comprising photolyase, endonuclease, alkylase, and/or 8-oxoguanine glycosylase. Another embodiment provides a composition comprising: an organic or inorganic sunscreen agent; a protein protection antioxidant comprising N-acetyl-S-(3,7,11-trimethyl-2E,6E,10-dodecatrienyl)-L-cysteine, L-carnosine, and/or L-ergothionine (i.e., one or more of antioxidant shown to have significant protein protection in one or more of the three assays described); and a DNA enzyme repair comprising photolyase, endonuclease, 8-oxoguanine glycosylase, and/or alkylase. A further embodiment comprises sunscreen agent comprising one, two, three, or more common sunscreen agents; a protein protection antioxidant comprising N-acetyl-S-(3,7,11-trimethyl-2E,6E,10-dodecatrienyl)-L-cysteine, L-carnosine, and L-ergothionine; and a DNA enzyme repair comprising photolyase, endonuclease, and 8-oxoguanine glycosylase. Some embodiments may modify these to include or substitute L-ergothioneine, L-carnosine, quercetin, resveratrol, epigallocatechin gallate, L-ascorbic acid, ferulic acid, and/or chlorogenic acid as protein protection antioxidants. In preferred embodiments of the invention, a plurality (2, 3, 4, 5, or more) of DNA and/or protein protection antioxidants, and/or a plurality (2, 3, 4, 5, or more) of DNA/RNA/protein repair enzymes, and/or a plurality (2, 3, 4, 5, or more) of sunscreens, may be used, i.e., 2-2-2, 2-3-2, 3-3-2, 3-4-2, 4-3-2, 4-4-2, 3-3-3, 4-5-2, 5-4-2, 5-3-2, 3-5-2, etc. Certain embodiments offer improved effects when larger weight percentages of each component are employed.

A pharmaceutically acceptable salt of the antioxidants or sunscreen agents may be used (substituted or used in combination) within the scope of the invention.

Compositions of the invention can exist in various forms. For example, the compositions of the invention can be in the form of a cream, a solution, a serum, a powder, an anhydrous preparation, an emulsion or microemulsion of the type water-in-oil (W/O) or of the type oil-in-water (O/W), a multiple emulsion, for example of the type water-in-oil-in-water (W/O/W), a gel, a solid stick, an ointment, or an aerosol. Certain embodiments administer the composition of the invention in encapsulated form, for example, in matrices of collagen and other conventional encapsulation materials (e.g., cellulose), in gelatin, in wax matrices, or as liposomal encapsulations. Embodiments of the invention add the inventive composition to aqueous systems or surfactant compositions for cleansing the skin.

Compositions of the invention may be anhydrous, even dry powders, however, generally preferred embodiments are aqueous, especially water and oil emulsions of the W/O or O/W variety. Water, when present, may be in amounts ranging from 5 to 90 wt. %, preferably from 35 to 65 wt. %, more preferably between 40 and 50 wt. %. Besides water, relatively volatile solvents may also be incorporated within compositions of the present invention. Certain embodiments preferably use one or more monohydric 1 to 3 carbon (i.e., C₁-C₃) alkanols. These include ethyl alcohol, methyl alcohol and isopropyl alcohol. The amount of monohydric alkanol may range from 5 to 50 wt. %, preferably from 15 to 40 wt. %, optimally between 25 to 35 wt. %, based on the total weight of the composition.

Collectively the water, solvents, silicones, esters, fatty acids, humectants, thickeners, viscosity modifiers, and/or other additives are viewed as acceptable carriers for the active agents of the invention. The total amount of carrier will range from roughly 1 to 99.9 wt. %, preferably from about 80 to 99 wt. %, based on the total weight of the composition.

Preservatives can desirably be incorporated into the cosmetic compositions of this invention to protect against the growth of potentially harmful microorganisms. Suitable traditional preservatives are known in the art and are preferably employed in amounts ranging from 0.01% to 2 wt. %, based on the total weight of the composition.

Inventive compositions may also contain, apart from the aforementioned, additives conventional in cosmetics, such as perfumes, thickeners, dyestuffs, deodorants, antimicrobial materials, back-fatting agents, complexing and sequestering agents, pearlescent agents, shampooing agents, plant extracts, vitamins, vitamin derivatives, and/or active ingredients.

### EXAMPLE 1

In an experiment illustrating the effect of an embodiment of the invention, 60 Caucasians with Fitzpatrick skin type I-II between the ages of 18 and 65 were included in a study. Subjects with a history of photodermatosis, skin cancer or actinic keratosis, and any active cancer or malignancy treated prior to the study were excluded, as were those with a history of inflammatory or atopic disorders, serious non-malignant disease (e.g., cardiovascular, pulmonary, systemic lupus erythematosus, scleroderma), those currently pregnant or lactating, or with psychiatric or addictive disorders. No subject was taking any photosensitizing or anti-inflammatory medications. All test materials were provided in barcoded, anonymized tubes. Both study participants and personnel were blinded to the applied product at the time of irradiations.

The study involved solar-simulated radiation produced by an ORIEL™ solar simulator (Model 81292, L.O.T. Oriel, Leatherhead, UK) containing a 1 kW xenon arc lamp with two dichroic mirrors, a collimator, and a 1-mm WG320 filter. The optical design of the ORIEL™ solar simulator gives a field of even irradiance (290-400 nm) at the skin surface when positioned 11 cm from the source, of which about 10% is UVB (280-320 nm) and the remainder UVA. The spectral irradiance was measured with an OL754™ spectroradiometer (Optronics, Orlando, Fla., USA), calibrated for wavelength and intensity against standard lamps. The spectroradiometer was used to calibrate a handheld IL700™ radiometer (International Light, Newburyport, Mass.), which was then used to rapidly monitor lamp output on a daily basis.

The minimal erythema dose (MED) was determined for each individual for solar-simulated UVR (290-400 nm) and expressed in mJ/cm² using a light-proof adhesive-backed foil template that were sequentially uncovered to deliver quantities of UV above and below the expected MED of skin phototype I-II individuals for solar-simulating UV radiation. The sites were examined 24 hours after irradiation and the MED was determined as the site that showed minimal, uniform perceptible erythema. Before irradiation, circular areas (10 mm diameter) were marked out on the nonexposed lower back of each participant, and 30 to 45 minutes before each experimental irradiation, each test product was applied to the circular irradiation areas (10 mm diameter, total area of 78.5 mm² each). When two products were applied, we allowed the first product to dry for 10 minutes before the application of the second product. A standard application thickness of 2 mg/cm² was used for each product, as this is the thickness used during FDA testing procedures for quantifying the SPF of traditional sunscreens. Then, on eight consecutive days, the experimental sites were exposed to solar-simulated UV radiation at 6 times MED. A negative control site received no solar-simulated UV radiation. 24 hours after the last exposure to solar-simulated UV radiation, skin specimens were obtained through a 4-mm punch biopsy from all sites for molecular analyses.

The skin biopsy specimens were cleaved in half, and one piece was thawed at room temperature, minced, and lysed by three cycles of freezing (in an ethanol-dry-ice bath) and thawing (at 95° C.). PC in cell lysates was measured by OXISELECT™ Protein Carbonyl ELISA Kit (Cell Biolabs, San Diego, Calif.) according to the manufacturer's instructions. For the measurements of CPD and 8OHdG, samples were digested for 12 hours at 60° C. with proteinase K in 100 mmol/L Tris-HCl (pH 7.4), 150 mmol/L NaCl, and 10 mmol/L EDTA (pH 8.0). Proteinase K was then heat inactivated at 95° C. for 10 minutes, and homogenates were extracted using the PUREGENE™ DNA isolation kit (Gentra Systems, Minneapolis, Minn., USA) containing two main reagents: cell lysis and protein precipitation solutions.

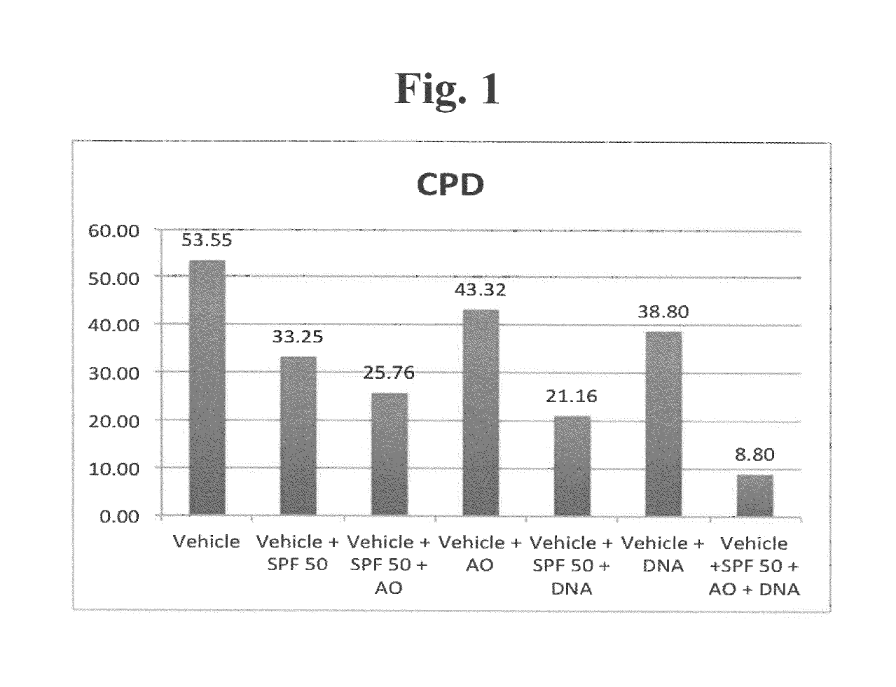

DNA was extracted from homogenates using a lysis buffer solution and then treated with RNase A. The kit removes proteins using a precipitation solution, followed by 2-propanol to pellet the DNA. Cyclobutane pyrimidine dimers (CPD) and 8-oxo-7,8-dihydro-2′-deoxyguanosine (8OHdG) were measured in duplicate and random order by specific ELISA kits [OXISELECT™ Cellular UV-Induced DNA Damage ELISA Kit (CPD), 96 assays (Cell Biolabs, San Diego, Calif., USA)] and OXISELECT™ Oxidative DNA Damage ELISA Kit (8-OHdG Quantitation, Cell Biolabs, San Diego, Calif., USA) according to the manufacturer's protocol. The results of cyclobutane pyrimidine dimers (CPD), 8-oxo-7,8-dihydro-2′-deoxyguanosine (8OHdG), and protein carbonylation (PC) measurements expressed in arbitrary units (amount of absorbance in ELISA relative to the untreated control, which was set as 1 by convention) are seen in FIG. 1-3. All laboratory analyses were made blinded to the irradiation protocol.

In the figures, it can be seen that a synergistic effect is realized for the combination of the sunscreen agent (A), the protein protection antioxidant complex (B), and the DNA repair enzyme complex (C) with a vehicle (V). The synergistic effect is based on comparing V+A+B vs. V+A to obtain the effect of adding B in the environment of A (i.e., δB), then comparing V+A+C vs. V+A to achieve the effect of adding C to the vehicle in the environment of A (i.e., δC), then comparing δB and δC, i.e., V+A+δB+δC, to the effect actually achieved by the combination of V+A+B+C.

As illustrated in FIG. 1, a composition comprising the vehicle comprising sunscreen showed 33.25 units of cyclobutane pyrimidine dimers (CPD) compared to 25.76 for a composition comprising the vehicle plus sunscreen and a protein protection antioxidant, or a 7.49 CPD reduction (P<0.05). A composition comprising the vehicle, sunscreen, and a DNA repair enzyme showed 21.16 CPD, or a 12.09 reduction from the vehicle comprising sunscreen (P<0.01). A composition comprising sunscreen, a protein protection antioxidant, and a DNA repair enzyme showed 8.80 CPD or a 24.45 reduction from the vehicle comprising sunscreen (P<0.001). If the effect of the composition comprising a protein protection antioxidant and a DNA repair enzyme were additive, then the result would be a 19.58 reduction in CPD. The actual 24.45 CPD reduction is multiplicative, providing an additional effect of 4.87 CPDs, or a 24.8% additional CPD reduction.

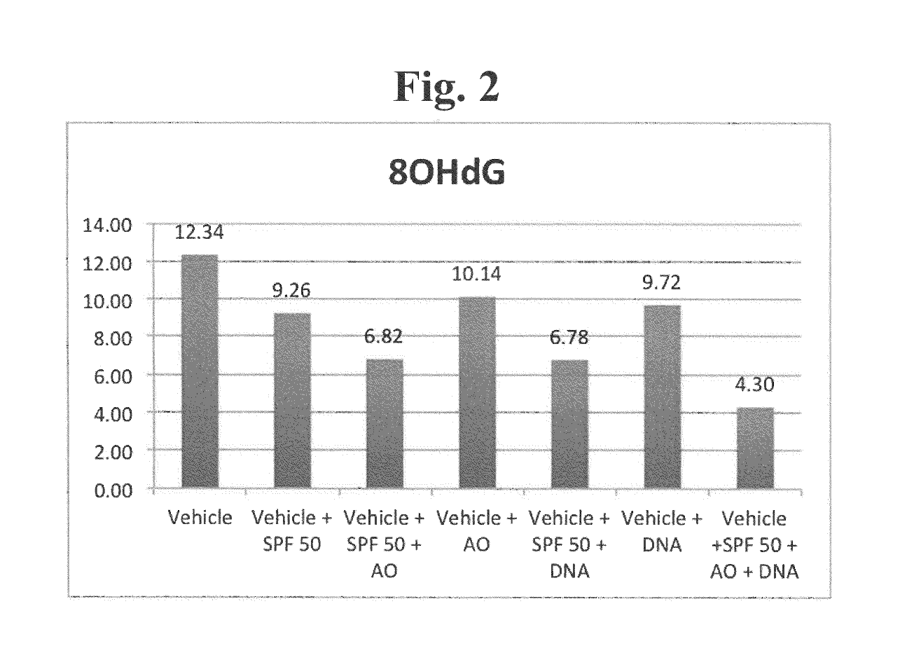

As illustrated in FIG. 2, a composition comprising the vehicle plus sunscreen showed 9.26 units of 8-oxo-7,8-dihydro-2′-deoxyguanosine (8OHdG) lesions compared to 6.82 for a composition comprising the vehicle plus sunscreen and a protein protection antioxidant, or a 2.44 8OHdG reduction (P<0.01). A composition comprising the vehicle, sunscreen, and a DNA repair enzyme showed 6.78 8OHdG lesions, or 2.48 reduction from a composition comprising the vehicle and sunscreen (P<0.01). A composition comprising the vehicle, sunscreen, a protein protection antioxidant, and a DNA repair enzyme showed 4.30 8OHdG lesions, or a 4.96 reduction from the vehicle plus sunscreen (P<0.001). The effect of the protein protection antioxidant and the DNA repair enzyme were additive in regard to 8-oxo-7,8-dihydro-2′-deoxyguanosine (8OHdG) lesions, not multiplicative, because a 2.48 reduction from the composition comprising the vehicle, sunscreen, and protein protection antioxidant plus a 2.44 reduction from the composition comprising vehicle, sunscreen, and a DNA repair enzyme equals 4.92, or approximately the same as the 4.96 reduction from the composition comprising the vehicle, sunscreen, a protein protection antioxidant, and a DNA repair enzyme.

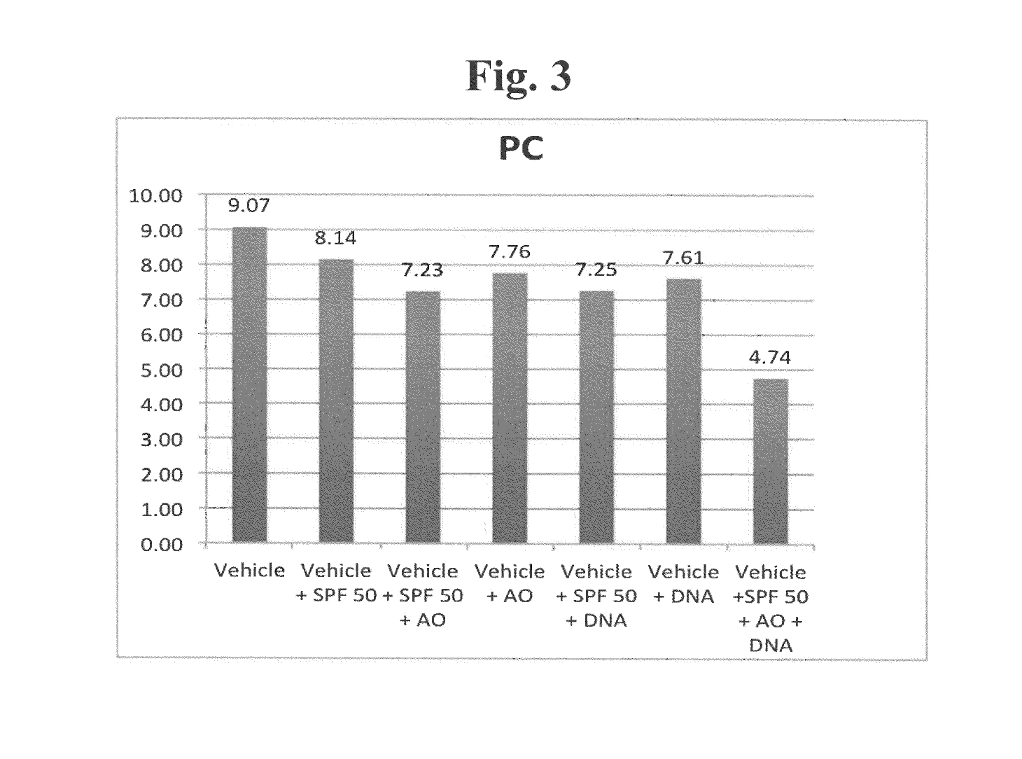

As illustrated in FIG. 3, reporting the mean amount of protein carbonylation (PC), a composition comprising the vehicle plus sunscreen showed 8.14 units of protein carbonylation (PC) compared to 7.23 for a composition comprising vehicle plus SPF 50 and AO complex, or a 0.91 PC reduction (P=n.s.). A composition comprising the vehicle, sunscreen, and a DNA repair enzyme showed 7.25 PCs, or 0.89 reduction from a composition comprising the vehicle plus sunscreen (P=n.s.). A composition comprising the vehicle, sunscreen, a protein protection antioxidant, and a DNA repair enzyme showed 4.74 PC or a 3.40 reduction from a composition comprising the vehicle plus sunscreen (P<0.001). If the effect of including a protein protection antioxidant and a DNA repair enzyme with the sunscreen were additive, the combination of a sunscreen, a protein protection antioxidant, and a DNA repair enzyme, would have had a 1.8 reduction in PC. The actual 4.74 PC reduction for the inventive composition is multiplicative and provides an addition effect of 4.87 PCs, or a 163.3% additional PC reduction.

Compositions according to the invention preferably have an additive, multiplicative, and/or synergistic effect, relative to its individual components, in protecting human skin against stress damage. Preferable compositions have an additive, multiplicative, and/or synergistic effect over the sunscreen SPF protection alone when measured by DNA and/or protein damage biomarkers.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B, and C” should be interpreted as one or more of a group of elements consisting of A, B, and C, and should not be interpreted as requiring at least one of each of the listed elements A, B, and C, regardless of whether A, B, and C are related as categories or otherwise. Moreover, the recitation of “A, B, and/or C” or “at least one of A, B, or C” should be interpreted as including any singular entity from the listed elements, e.g., A, any subset from the listed elements, e.g., A and B, or the entire list of elements A, B, and C.

1. A composition, comprising:
a sunscreen agent;
a plurality of DNA repair enzymes; and
a plurality of DNA and/or protein protective antioxidants.

2. The composition of claim 1, having an SPF of at least 15.

3. The composition of claim 1, wherein the plurality of DNA and/or protein antioxidants comprises an amino acid, a derivative of an amino acid, a peptide, a derivative of a peptide, or a mixture of two or more of any of these.

4. The composition of claim 1, wherein the plurality of DNA and/or protein protective antioxidants comprise quercetin, resveratrol, epigallocatechin gallate, L-ascorbic acid, ferulic acid, and chlorogenic acid, N-acetyl-S-(3,7,11-trimethyl-2E,6E,10-dodecatrienyl)-L-cysteine, L-carnosine, L-ergothionine, or a mixture thereof.

5. The composition of claim 1, wherein the plurality of DNA and/or protein protective antioxidants comprises N-acetyl-S-(3,7,11-trimethyl-2E,6E,10-dodecatrienyl)-L-cysteine, L-carnosine, L-ergothionine, or a mixture thereof.

6. The composition of claim 1, wherein the plurality of DNA and/or protein antioxidants comprises N-acetyl-S-(3,7,11-trimethyl-2E,6E,10-dodecatrienyl)-L-cysteine.

7. The composition of claim 1, wherein the plurality of DNA and/or protein antioxidants comprises L-carnosine.

8. The composition of claim 1, wherein the plurality of DNA and/or protein antioxidants comprises
L-ergothionine and
N-acetyl-S-(3,7,11-trimethyl-2E,6E,10-dodecatrienyl)-L-cysteine, L-carnosine, or both.

9. The composition of claim 1, wherein the plurality of DNA and/or protein antioxidant comprises N-acetyl-S-(3,7,11-trimethyl-2E,6E,10-dodecatrienyl)-L-cysteine, L-carnosine, and L-ergothionine.

10. The composition of claim 1, wherein the antioxidant is a protein protection antioxidant.

11. The composition of claim 1, wherein the antioxidant is a DNA protection antioxidant.

12. The composition of claim 1, wherein the plurality of DNA repair enzymes comprises photolyase, endonuclease, 8-oxoguanine glycosylase, or a mixture thereof.

13. The composition of claim 1, wherein the plurality of DNA repair enzymes comprises photolyase.

14. The composition of claim 1, wherein the plurality of DNA repair enzymes comprises endonuclease.

15. The composition of claim 1, wherein the plurality of DNA repair enzymes comprises 8-oxoguanine glycosylase.

16. The composition of claim 1, wherein the plurality of DNA repair enzymes comprises alkylase.

17. The composition of claim 1, wherein the plurality of DNA repair enzymes comprises photolyase, endonuclease, and 8-oxoguanine glycosylase.

18. The composition of claim 1, wherein the sunscreen agent is present in an amount of 1-40 wt. %, based on a total weight of the composition.

19. The composition of claim 1, wherein the plurality of DNA and/or protein protective antioxidants is present in an amount of 0.01-25 wt. %, based on a total weight of the composition.

20. The composition of claim 1, wherein the plurality of DNA repair enzymes is present in an amount of 10⁻⁹ to 5 wt. %, based on a total weight of the composition.

21. A method of making the composition of claim 1, the method comprising:
combining a sunscreen agent, a DNA repair enzyme, and a DNA and/or protein protective antioxidant.

22. A method of improving skin health, the method comprising:
topically applying to human skin the composition of claim 1,
wherein the improving includes superior prevention of certain genetic biomarkers, antioxidative protein protection, and/or sun protection factor, to a degree greater than an additive effect of individual components of the composition, normalized for presence of all components in the composition.

23. A method of improving a protective effect of a topical skin formulation, the method comprising:
combining a sunscreen agent, two or more DNA repair enzyme, and two or more DNA and/or protein protective antioxidants, in any order, to obtain a composition
wherein the composition has the protective effect, which is greater than an additive effect of individual components of the composition, normalized for presence of effective components in the composition.
